(19) United States
(12) Patent Application Publication
(10) Pub. No.: US 2022/0409044 A1
(43) Pub. Date: Dec. 29, 2022

(54) SYSTEM AND METHOD FOR MEASURING AT LEAST ONE PARAMETER OF EYE

(71) Applicant: ICARE FINLAND OY, Vantaa (FI)

(72) Inventor: Mika SALKOLA, Espoo (FI)

(73) Assignee: ICARE FINLAND OY, Vantaa (FI)

(21) Appl. No.: 17/778,928

(22) PCT Filed: Nov. 20, 2020
(86) PCT No.: PCT/FI2020/050786

§ 371 Date: May 23, 2022

(30) Foreign Application Priority Data
Nov. 29, 2019 (FI) 20196032

(51) Int. Cl. A61B 3/10; A61B 3/00; A61B 3/16

(52) U.S. Cl. CPC A61B 3/1005; A61B 3/0025; A61B 3/16

(57) ABSTRACT

A system for measuring at least one parameter of an eye. The system includes a probe detachably arranged within a housing, wherein the probe is operable to impact a surface of the eye with a predefined impact attribute, at least one coil operable to maintain the probe within the housing, to release the probe towards the surface of the eye and to retract the probe into the housing, a probe vibration means operable to induce vibration to the probe, a measuring means for measuring a change in vibration of the probe upon impact on the surface of the eye and a controller configured to use the measured change in vibration of the probe to determine the at least one parameter of the eye.

### TECHNICAL FIELD

The present disclosure relates generally to medical devices; and more specifically, to medical devices for measuring at least one parameter of eye. Moreover, the present disclosure relates to methods for measuring at least one parameter of eye.

### BACKGROUND

Notably, global prevalence of eye diseases is estimated to be more than 1 billion. Specifically, eye diseases can be treated and/or prevented by accurate diagnosis of an abnormality in any parameter of an eye. In an example, disease, such as, glaucoma can be diagnosed and/or treated by regular monitoring of fluid pressure inside the eye. The thickness of cornea of the eye is related to prevalence of glaucoma and is an indication of several other ocular diseases. It will be appreciated that glaucoma causes intrinsic deterioration of optic nerve of the eye owing to high fluid pressure inside the eye, often leading to permanent vision loss. Therefore, monitoring parameters of the eye on regular basis is essential to identify an abnormality in the eye.

Conventionally, devices such as tonometer and pachymeter are employed for measurement of the parameters of the eye. In this regard, a tonometer is used to measure fluid pressure inside the eye, namely, Intraocular Pressure (IOP). The measured fluid pressure inside the eye enables an examiner to determine a risk of, for example, glaucoma. Moreover, a pachymeter is used to measure thickness of cornea of the eye, wherein such corneal pachymetry is essential prior to refractive surgery, prior to Limbal Relaxing Incision (LRI) surgery, for keratoconus screening, for glaucoma screening, and so forth.

However, conventional devices for measurement of the parameters of the eye to identify occurrence of any diseases, for example, glaucoma, are not reliable and swift. Typically, readings from a pachymeter results in false high-pressure reading for an eye in case of thick cornea of the eye and in false low-pressure reading for an eye in case of thin cornea of the eye. Subsequently, the pachymeter is not reliable and only creates a baseline for comparison with future tests to be performed on the eye, for example, tonometer test, dilated eye test, visual field test, imaging test and gonioscope.

Moreover, conventional tonometer imprecisely measures Intraocular Pressure (IOP) inside the eye and merely provides an estimated reading with great deal of noise. In this regard, many factors (for example, technique of tonometer, calibration of tonometer, corneal curvature, corneal hydration, corneal thickness, corneal rigidity, and so forth) affect reading of the tonometer. Moreover, conventional tonometer does not eliminate noise in the reading owing to the aforesaid factors, thereby making the conventional tonometer inaccurate and unreliable for efficient measurement of Intraocular Pressure inside the eye. Additionally, inspection of the eye using tonometer requires anesthetic operations to be performed prior to the inspection. Such inspection of the eye may be painful and further cause discomfort and irritation for patients.

The conventional devices for inspection of the eye are time-intensive, less sensitive and prone to errors. Moreover, readings from conventional devices require great amount of human intervention for identifying an abnormality in the eye.

Therefore, in light of the foregoing discussion, there exists a need to overcome the drawbacks associated with conventional devices used for measurement of parameters of eyes.

### SUMMARY

The present disclosure seeks to provide a system for measuring at least one parameter of eye. The present disclosure also seeks to provide a method for measuring at least one parameter of eye. The present disclosure seeks to provide a solution to the existing problem of conventional medical devices that inaccurately measure the at least one parameter of eye thereby affecting diagnosis of diseases in the eye, and further causing discomfort for patients during the measurement. An aim of the present disclosure is to provide a solution that overcomes at least partially the problems encountered in prior art, and provides a medical device that accurately measures the at least one parameter of the eye for efficient diagnosis of diseases.

In one aspect, an embodiment of the present disclosure provides a system for measuring at least one parameter of an eye, the system comprising a probe detachably arranged within a housing, wherein the probe is operable to impact a surface of the eye with a predefined impact attribute; at least one coil operable to maintain the probe within the housing, to release the probe towards the surface of the eye and to retract the probe into the housing; a probe vibration means operable to induce vibration to the probe; a measuring means for measuring a change in vibration of the probe upon impact on the surface of the eye; and a controller configured to use the measured change in vibration of the probe to determine the at least one parameter of the eye.

In another aspect, an embodiment of the present disclosure provides a method for measuring at least one parameter of an eye, the method comprising arranging a probe to impact a surface of the eye with a predefined impact attribute and a predefined vibration; measuring impact attribute of the probe and vibration of the probe, during impact on the surface of the eye; calculating, using at least one of: the predefined impact attribute, the predefined vibration, the measured impact attribute, the measured vibration, a change in the vibration of the probe, upon impact on the surface of the eye; and determining, using the change in the vibration of the probe, the at least one parameter of the eye.

Embodiments of the present disclosure substantially eliminate or at least partially address the aforementioned problems in the prior art, and enables swift and accurate measurement of the at least one parameter of the eye that is free of noise, in a painless manner; and further enables reliable diagnosis of disease without involving numerous tests thereby considerably saving cost and time associated with various tests for a patient.

Additional aspects, advantages, features and objects of the present disclosure would be made apparent from the drawings and the detailed description of the illustrative embodiments construed in conjunction with the appended claims that follow. It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those in the art will understand that the drawings are not to scale. Wherever possible, like elements have been indicated by identical numbers.

Embodiments of the present disclosure will now be described, by way of example only, with reference to the following diagrams wherein:

FIG. 1 is a schematic illustration of a system for measuring at least one parameter of an eye, in accordance with an embodiment of the present disclosure;

FIG. 2 is a schematic illustration of a system for measuring at least one parameter of an eye, in accordance with an exemplary embodiment of the present disclosure;

FIG. 3 is a schematic illustration of a system for measuring at least one parameter of an eye, in accordance with an exemplary embodiment of the present disclosure;

FIG. 4 is a schematic illustration of a system for measuring at least one parameter of an eye, in accordance with an exemplary embodiment of the present disclosure;

FIG. 5A is a graphical representation of change in speed of a probe with respect to change in time, in accordance with an embodiment of the present disclosure;

FIG. 5B illustrates the frequency change profile upon probe impact on the surface of the eye, in accordance with an embodiment of the present disclosure;

FIGS. 6A, 6B, 6C and 6D are schematic illustrations of movement of a probe with respect to a surface of an eye, in accordance with an embodiment of the present disclosure;

FIG. 7 illustrates steps of a method for measuring at least one parameter of an eye, in accordance with an embodiment of the present disclosure;

FIG. 8 illustrates the waveform of standing wave of the probe on an impact with the cornea, in accordance with an embodiment of the present disclosure; and

FIGS. 9A, 9B, 9C, 9D and 9E are schematic illustrations of propagations of the waves in a probe during the impact of the cornea, in accordance with an embodiment of the present disclosure.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practicing the present disclosure are also possible.

In one aspect, an embodiment of the present disclosure provides a system for measuring at least one parameter of an eye, the system comprising a probe detachably arranged within a housing, wherein the probe is operable to impact a surface of the eye with a predefined impact attribute; at least one coil operable to maintain the probe within the housing, to release the probe towards the surface of the eye and to retract the probe into the housing; a probe vibration means operable to induce vibration to the probe; a measuring means for measuring a change in vibration of the probe upon impact on the surface of the eye;

and a controller configured to use the measured change in vibration of the probe to determine the at least one parameter of the eye.

In another aspect, an embodiment of the present disclosure provides a method for measuring at least one parameter of an eye, the method comprising arranging a probe to impact a surface of the eye with a predefined impact attribute and a predefined vibration; measuring impact attribute of the probe and vibration of the probe, during impact on the surface of the eye; calculating, using at least one of the predefined impact attribute, the predefined vibration, the measured impact attribute, the measured vibration, a change in the vibration of the probe, upon impact on the surface of the eye; and determining, using the change in the vibration of the probe, the at least one parameter of the eye.

The system for measuring at least one parameter of the eye as described in the present disclosure provides a patient friendly, quick and painless solution to detect different conditions and diseases of the eye. Specifically, the present disclosure provides a device for measuring at least one parameter of the eye, wherein the device comprises a probe that impacts the surface of the eye with predefined impact attributes (for example predefined speed) and predefined vibration (predefined frequency, pulse of vibration, waveform related to vibration). The device further operates to calculate a change in vibration from the predefined vibration so as to determine the at least one parameter. It will be appreciated that the at least one parameter is an indicative of a condition or illness in the eye or a physical parameter of the eye. Notably, the system accurately measures the at least one parameter for holistic inspection of the eye that accounts for less time, money and discomfort for patients. Moreover, accurate and on-time diagnosis of any condition of the eye using the system as described herein enables curing the condition effectively or prevents the eye from further damage, and further prevents cases of permanent vision loss due to diseases like glaucoma. A time within which the probe impacts the surface of the eye and retracts therefrom, is lesser than a reaction time of the eye thereby causing minimum discomfort to the eye of patient. Moreover, the probe is coated with biological covering to prevent any pain in the eye and/or damage on the surface of the eye due to, for example, scratching of the surface of the eye due to impact of the probe. Furthermore, the system is light weighted thereby enabling ease of use thereof by an examiner. Moreover, the probe touches the surface of the eye at a low speed to prevent any damage to the surface of the eye. Furthermore, beneficially, a low amount of energy is required to operate the system owing to light weight and low impact speed thereof. Beneficially, the system measures the at least one parameter of the eye without any negative effect, thereby ensuring comfort and safety of the patient.

The present disclosure provides a system for measuring the at least one parameter of the eye. In this regard, the measurement of the at least one parameter of the eye by the system is indicative of a condition of the eye, for example, normal condition, an illness, a stage of the illness, an abnormal condition, and the like. Optionally, the at least one parameter of the eye is thickness of cornea of the eye, pressure of the eye, corneal water content. More optionally, the at least one parameter of the eye is used to diagnose an abnormality associated with corneal thickness (for example, ocular hypertension, glaucoma), corneal opacity (for example, cataract, corneal ulcer), Intraocular Pressure (TOP) within the eye, and the like.

It will be appreciated that a deviation from normal measurements associated with the at least one parameter of the eye is regarded as an illness or an abnormal condition. Subsequently, the system accurately measures the at least one parameter of the eye that enables identification of an illness or abnormality in the eye of a patient based on a measured value of a parameter and normal measurement value associated with the parameter.

Optionally, the identification of the illness or abnormality in the eye of the patient is performed manually by a user of the system, for example, a doctor, an ophthalmologist, an optometrist, a technician, and the like or patient him or herself. In this regard, readings associated with measurement of the at least one parameter are used to identify the illness or abnormality. More optionally, the identification of the illness or abnormality in the eye of the patient is performed automatically by the controller (as described in detail later, herein) in the system.

The system comprises the probe detachably arranged within the housing. Specifically, the probe is an elongate instrument used for impacting the surface of the eye. In this regard, the probe is operable to impact the surface of the eye with the predefined impact attribute. Furthermore, a vibration in the probe is induced by at least one probe vibration means. Pursuant to embodiments of the present disclosure, the probe is an elongate bar having a first end and a second end. Moreover, the first end of the probe has a spherical protrusion, wherein the spherical protrusion is the part that impacts the surface of the eye. Additionally, optionally, the second end of the probe is suspended within the housing.

Optionally, the probe is a metallic rod. In an instance, a ferromagnetic material (for example, iron, nickel, and the like) is used for manufacturing the probe or elastomer with ferromagnetic compound. In another instance, a piezoelectric material, for example, quartz, is used for manufacturing the probe. In yet another instance, a combination of ferromagnetic material and piezoelectric material is used for manufacturing the probe. Moreover, optionally, a surface of the probe is made up of or covered at least partially with bio-compatible material. Beneficially, such bio-compatible covering of the probe enables the system to function in intimate contact with living tissues of the eye causing minimal discomfort or pain. Furthermore, a thickness of the probe is in a range of 0.1 mm to 1 mm, for example 0.3 mm. Moreover, the probe is very lightweight. In an example, the weight of the probe is 0.25 milligrams (mg).

Specifically, the “housing” refers to a protective covering that substantially encases components of the system (namely, the probe, the measuring means, the probe vibration means, and the controller). Moreover, optionally, the housing is provided with apertures (namely, inlet and/or outlet) arranged thereon. Typically, the apertures are operable for enabling supply of electrical power to the components of the system. Additionally, optionally, the housing is designed strategically for making the system convenient for use, and easy to handle and hold.

Moreover, the probe is detachably arranged within the housing. In this regard, the probe is arranged to move along a longitudinal axis of the housing to impact the surface of the eye. Optionally, the housing completely encompasses the probe. Alternatively, optionally, a portion (for example, the spherical protrusion) of the probe is outside the housing. In an instance, the housing is manufactured using a polymer. In another instance, the housing is manufactured using a metal alloy.

Furthermore, the probe is operable to impact the surface of the eye with predefined impact attributes. In this regard, the probe strikes the surface of the eye while exerting force thereon. Pursuant to embodiments of the present disclosure, the probe touches the surface of the eye gently and further exerts a force onto the surface of the eye, causing the surface of the eye to bend inwards. In this regard, the probe has sufficiently large surface area to prevent piercing through the surface of the eye, thereby eliminating an instance of damage of tissues of the surface of the eye. Moreover, the predefined impact attributes are the characteristic features of the probe with which the probe impacts the surface of the eyes. The probe hits on the corneal surface and bounces back from the surface of the cornea, and at the time of the impact some of the vibration of the probe goes towards the cornea and then part of the vibration reflects back from the endothelium of the cornea.

Optionally, the impact attribute of the probe is at least one of: speed of the probe, kinetic energy of the probe. It will be appreciated that the speed of the probe is a speed with which the probe is moving towards the eye. Moreover, the predefined speed of the probe is a speed of the probe when the probe moves towards the surface of the eye and at an instance of first contact of the probe with the surface of the eye (namely, impact moment). In an example, the speed of the probe is in a range of 0.20 metres per second (m/s) to 0.35 m/s. Notably, the speed of the probe is low thereby ensuring minimal energy is required to drive the probe and eliminating an instance of damage of the tissues of the surface of the eye.

The system comprises the at least one coil operable to maintain the probe within the housing, to release the probe towards the surface of the eye and to retract the probe into the housing. It will be appreciated that the at least one coil is an electric conductor (for example, a wire) in the shape of a coil, spiral or helix. Specifically, the at least one coil is placed inside the housing of the system, such that the at least one coil surrounds the probe. Notably, the probe running through the at least one coil is energized as the system is turned ON, whereby the at least one coil decompresses to release the probe towards the surface of the eye. Additionally, the at least one coil compresses to retract the probe into the housing after the probe impacts the surface of the eye. Subsequently, the at least one coil maintains the probe within the housing. In an instance, there are two coils in the system, wherein the two coils surround the probe at two locations on the probe, and wherein the two locations of the coils of the probes are non-overlapping.

Optionally, the at least one coil is an electromagnetic coil. More optionally, the at least one coil induces an electric field and/or a magnetic field into the probe for vibration thereof. Moreover, optionally, the at least one coil is arranged within a coil frame, wherein the coil frame is a skeleton structure that holds the at least one coil in desired manner.

The system comprises a probe vibration means operable to induce vibration to the probe. The induced vibration can be for example, a frequency in the range of 0.5 kilo Hertz (kHz) to 100 MHz. As an further example the frequency can be in range from 0.5, 1, 5, 10, 15, 20, 25, 30, 35, 40, 45, 50, 55, 60, 65, 70, 75, 80, 85, 90, 95 or 100 kHz or 1, 5, 10, 15, 20, 25, 30, 35, 40, 45, 50, 55, 60, 65, 70, 75 or 80 MHz up to 5, 10, 15, 20, 25, 30, 35, 40, 45, 50, 55, 60, 65, 70, 75, 80, 85, 90, 95, or 100 kHz or 1, 5, 10, 15, 20, 25, 30, 35, 40, 45, 50, 55, 60, 65, 70, 75, 80, 85, 90, 95 or 100 MHz.

Optionally the vibration can be at least one of: a continuous vibration, a standing wave vibration, a pulsed vibration, a vibration comprising two or more vibration frequencies. Furthermore, the induced vibration can be a single frequency or multiple frequency vibration. As a further example the induced vibration can be for a standing wave type of vibration. An example of a pulsed vibration is to induce a vibration pulse from for example middle part of the probe. The vibration pulse would in such setup move towards ends of the probe with characteristic speed of sound in the probe. Further the induced vibration can be a combination of two or more frequencies of vibration at the same time (to procure interference patterns). As a further example the induced vibration can have predefined waveform of the vibration. Furthermore, amplitude of the induced vibration can be determined while inducing the vibration.

Optionally, the probe vibration means is calibrated previously, to induce vibration to the probe. Optionally, the probe vibration means comprises at least one of: a rnagnetostrictive oscillator, a piezoelectric oscillator, a transducer, an amplifier, a multivibrator. In an example, the probe vibration means employs a rnagnetostrictive oscillator to induce vibration to the probe. In this regard, the at least one coil is implemented using two coils (namely, a first coil L₁ and a second coil L₂), wherein the two coils are employed to surround the probe. The two coils surrounding the probe form an alternating magnetic field parallel to the longitude of the probe when supplied with electrical energy. The first coil and the second coil are connected with each other, via a transistor, for example a Field-Emitting Transistor (FET), Bipolar Junction transistor (BJT) and the like. The first coil (L₁) is further connected, using connecting wires, to a variable capacitor (C) and an electrical power supply, (for example, a battery) to form a collector circuit with the transistor. Moreover, the second coil is connected to form a base circuit with the transistor. The collector circuit oscillates with a given frequency when the electrical power is supplied thereto (i.e. electrical power supply is turned ON), wherein the given frequency is defined by:

f=1/(2π√(L₁ C)).

Moreover, alternating current flowing through the first coil L₁ produces an alternating magnetic field along the longitude of the probe. Subsequently, the probe starts to vibrate longitudinally due to magnetostrictive effect. Typically, a frequency of the oscillator circuit (namely, the given frequency), and further strength of the alternating magnetic field and frequency of the vibration of the probe, is controlled using the variable capacitor. It will be appreciated that the vibration induced in the probe is dependent on the strength of the alternating magnetic field, a nature of material of the probe. In this regard, the frequency of vibration of the of probe induced by the alternating magnetic field is defined by:

f=1/2 [symbol] √(Y/ρ),

wherein [symbol] is a length of the probe,

Y is Young's modulus, and

ρ is a density of the material of the probe.

The base circuit of the second coil acts as a feed-back coil to the probe. Optionally, the variable capacitor is calibrated in a manner that the frequency of the oscillator circuit induces predefined vibration to the probe. In another embodiment the vibration is induced to the probe synthetically by modulating frequency pulse or generating the pulses otherwise.

In another example, the probe vibration means employs a piezoelectric oscillator to induce the vibration to the probe. In this regard, the probe is manufactured using piezoelectric material, for example, a metal, quartz crystals, or a combination thereof. Moreover, the probe is connected to a primary winding of a transformer, wherein the primary winding of the transformer is inductively coupled to an electronic oscillator. The electronic oscillator is a base turned oscillator circuit. Moreover, a secondary winding of the transformer has two coils (namely, a first coil L₁ and a second coil L₂), wherein the first coil L₁ is shunted with a variable capacitor forming a base circuit with a transistor of the oscillator and the second coil L₂ is connected with an electrical power supply further forming a collector circuit with the transistor of the oscillator. The first coil L₁ and the second coil L₂ of the oscillator circuit are inductively coupled. Moreover, the oscillator circuit produces high frequency alternating voltage upon supplying electrical power to the second coil (i.e. upon turning the electrical power supply ON). Subsequently, an oscillatory Electromotive force (EMF) is induced in the primary winding of the transformer owing to transformer action. Herein, the probe is induced with vibration owing to an inverse piezo-electric effect that sets the piezoelectric material in the probe to vibrate. Additionally, the high frequency alternating voltage is fed to the probe. In this regard, a frequency of vibration of the probe is given by:

f=P/2 [symbol] √(Y/ρ),

wherein P=1, 2, 3, . . . for fundamental, first overtone, second overtone,

[symbol] is a length of the probe,

Y is Young's Modulus, and

ρ is a density of piezoelectric material in the probe.

It will be appreciated that the variable capacitor needs to be changed to change the frequency of the alternating voltage, and thus frequency of vibration induced in the probe.

More optionally, the transducer operates to convert electrical power from an electrical power supply to mechanical vibrations. In an example, the transducer is an electromechanical transducer that operates by utilizing piezoelectricity, magnetostriction or electrostriction. Moreover, optionally, the transducer is an oscillator. Additionally, the amplifier operates to amplify the frequency of vibration of the probe by amplifying, for example, alternating magnetic field, alternating voltage, alternating electric field, and the like, that induces vibration in the probe. Furthermore, the multivibrator may be designed to implement an oscillator circuit that induces vibration to the probe. Furthermore, optionally, a frequency of the induced vibration of the probe is in a range of 0.5 kilo Hertz (kHz) to 100 Mega Hertz (MHz) as discussed above. In this regard, the frequency of the vibration of the probe refers to a rate and an amplitude with which the probe vibrates. Moreover, the probe vibration means induces the vibration to the probe with a specific wave form.

The system comprises a measuring means for measuring a change in vibration of the probe upon impact on the surface of the eye. It will be appreciated that the vibration (namely, predefined vibration) induced in the probe by the probe vibration means has a specific waveform and the predefined frequency.

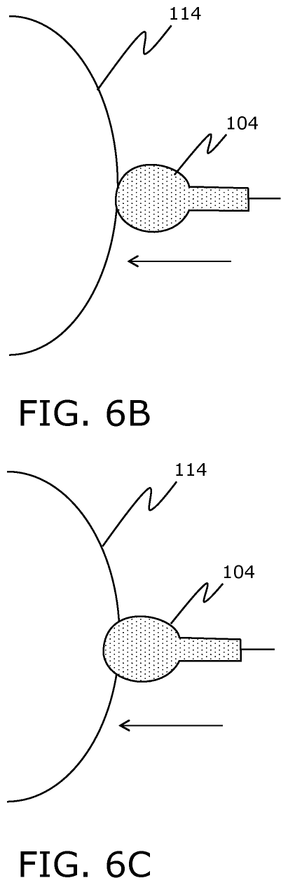

The frequency or waveform of the vibration of the probe can change from a first initial value to a second end value, wherein the end value can be the same as, higher or lower than the first initial value. During the impact on the surface of the eye or duration of the movement of the probe, the frequency of the vibration of the probe may get lower or it may get higher compared to the first initial value of the frequency of the vibration of the probe. For example, if the probe hits the surface of the eye which has a harder surface, then the frequency is likely to increase from the first initial value (as indicated in the figure FIG. 5B 0.9 to 1.0 of relative values). Measuring a change of the frequency of vibration or waveform of the probe and a profile of frequency change can be used for example as an indicator of a parameter of an eye. Frequency (arbitrary units) refers to the relative values, i.e. 1 can be same as 5 kHz to 100 MHz etc.

A part of the induced probe vibration reflects back from the outer surface of cornea, epithelium and a part of the induced probe vibration propagates through corneal tissue and reflects back from the inner surface of cornea, endothelium. These reflections of the vibration are mixed with the originally induced frequency or waveform of the vibration.

It will be appreciated that the measuring means measures the vibration of the probe, as soon as the system is operated, i.e., when the probe starts to move towards the surface of the eye, impacts the surface of the eye, exerts a force onto a surface of the eye, and retracts from the surface of the eye towards the housing. Moreover, optionally, the measuring means measures the vibration of the probe continuously or instantaneously.

Optionally, the measuring means comprises at least one of: a transducer, an accelerometer, a speed sensor, a frequency sensor. In this regard, the transducer converts the vibration of the probe to electrical energy so as to measure a change in the vibration of the probe. Alternatively, the measuring means employs the accelerometer to measure a change in vibration of the probe. Moreover, the speed sensor measures instantaneous speed of the probe. The speed of the probe is measured during movement of the probe towards the surface of the eye, impact on the surface of the eye, exertion of force on the surface of the eye and retraction of the probe from the surface of the eye. Additionally, the frequency sensor measures frequency of the impact of the probe on the surface of the eye for any future reference and/or calculation.

Furthermore, optionally, characteristic attributes of the at least one coil are measured to calculate the change in vibration of the probe. In an instance, when the probe vibration means is a magnetostrictive oscillator, then attributes (for example, length, pitch, and the like) of the feed-back coil (namely, the second coil forming the base circuit) is measured to measure the change in the vibration of the probe. In an embodiment, additional one or more excitation coils can be used to reach the supersonic frequencies, wherein the same or different coils are used to measure frequency change and reflected signals.

Furthermore, the predefined impact attributes of the probe and impact attributes measured during the impact can be employed to when determining a change in the vibration of the probe. For sake of clarity, change in vibration is explained in terms of frequency in conjunction with FIG. 5B.

The change in the vibration can be considered for example as interference patterns of the probe, changes in the waveform of the vibration in the probe, amplitude of the vibration of the probe, time differences between the reflection pulses.

It will be appreciated that such change in the speed of the probe and/or change in the vibration of the probe, during the impact on the surface of the eye, is observed due to reflection of different phases, namely, the damping effect of touch.

The system comprises a controller configured to use the measured change in vibration to determine the at least one parameter of the eye. Typically, the controller manages, commands, directs or regulates operations of other devices using control loops. Pursuant to embodiments of the present disclosure, the controller governs operation of the components of the system, namely, the probe, the probe vibrating means, and the measuring means. Moreover, the change in vibration of the probe is analyzed by the controller to determine the at least one parameter of the eye. In this regard, readings from the measuring means relating to the change in vibration of the probe are acquired by the controller and further analyzed to determine the at least one parameter. The at least one parameter is an indicative of a condition and/or an abnormality of the eye. In an example, a change in vibration of the probe is analyzed by the controller to determine thickness of cornea of the eye. In an example, a change in vibration of the probe is analyzed by the controller to determine Intraocular pressure inside the eye. Optionally, the controller is a computing unit. As an example of measurement is to measure/determine a wave form of the vibration on the probe before, during and after the impact with the eye. The waveform can be used to calculate for example parameter relating the thickness of the cornea by measuring distance of propagating vibrational pulses in the probe after the impact. Indeed, according to one example a waveform of the vibration can be measured as a function of time along the probe or at a certain point (or length) of the probe.

Optionally, the controller comprises at least one of: a network adapter, a memory unit, a processor. More optionally, the controller is capable of communicating readings acquired from the components of the system to a user device, for example, a mobile phone, a computer, and the like. Such communication of the readings to the user device enables a user to further perform analysis on the readings to draw conclusions and inferences relating to the eye and/or the at least one parameter. Moreover, optionally, the controller is capable of acquiring data from an external database to perform analysis on the readings (namely, change in vibration of the probe) to determine the at least one parameter, and further analysis on the at least one parameter to determine condition, risk, disease and abnormality associated with the eye. It will be appreciated that the controller communicates with the user device and/or the external database via a data communication network, for example, Internet.

Optionally, the components of the system are powered using electrical power supply from, for example, an electrical socket, at least one electrical battery, and the like.

According to an example it will be appreciated that the tissues of the surface of the eye reflexes after 0.2 seconds. Moreover, a time between the impact moment by the probe on the surface of the eye and complete retraction of the probe from the surface of the eye is 0.1 seconds thereby enabling operation of the system before reflexing of the tissues. Furthermore, the measuring means takes 0.05 seconds to reliably measure the change in vibration of the probe.

Moreover, optionally, in order to minimize error in measurement of change in vibration and to accurately measure the change in vibration, procedure of impacting the surface of the eye with the probe is performed multiple times, for example 6 times for the eye. Therefore, such repeated measurement removes inaccuracy and error in the reading of the system thereby enabling reliable determination of the at least one parameter of the eye and further diagnosis of a disease in the eye.

The present disclosure also relates to the method as described above. Various embodiments and variants disclosed above apply mutatis mutandis to the method.

Optionally, a frequency of the induced vibration of the probe is in a range of 0.5 kilo Hertz (kHz) to 100 Mega Hertz (MHz). Optionally, the at least one parameter of the eye is any one of: thickness of cornea of the eye, pressure of the eye, corneal water content. Optionally, the impact attribute of the probe is at least one of: speed of the probe, kinetic energy of the probe.

### DETAILED DESCRIPTION OF THE DRAWINGS

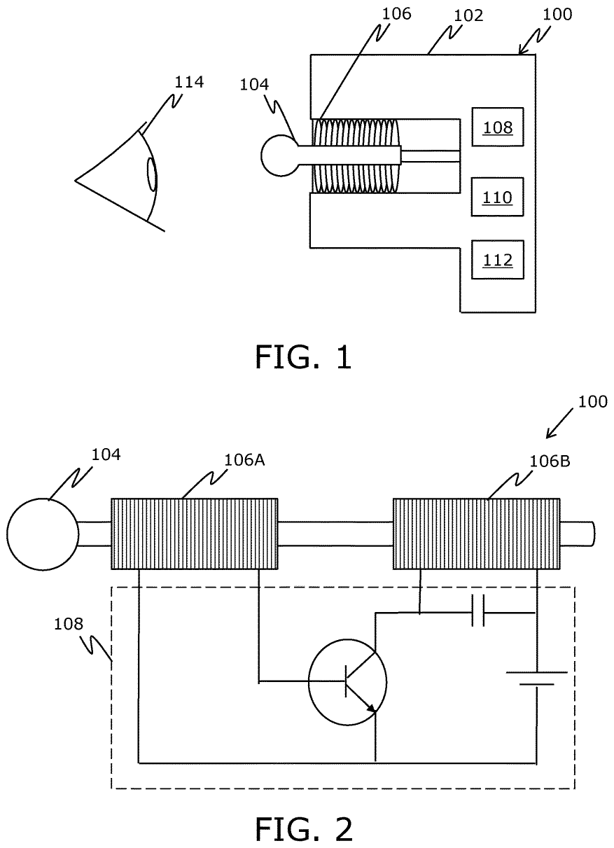

Referring to FIG. 1, there is shown a schematic illustration of a system 100 for measuring at least one parameter of an eye, in accordance with an embodiment of the present disclosure. As shown, the system 100 comprises a probe 104 detachably arranged within a housing 102, a coil 106, a probe vibration means 108, a measuring means 110 and a controller 112. The probe 104 is operable to impact a surface 114 of the eye with a predefined impact attribute. Additionally, the coil 106 is operable to maintain the probe 104 within the housing 102. Moreover, the coil 106 is operable to release the probe 104 towards the surface 114 of the eye and to retract the probe 104 into the housing 102. The probe vibration means 108 is operable to induce vibration to the probe 104 and the measuring means 110 is operable to measure a change in vibration of the probe 104 upon impact on the surface of the eye. The controller 112 is configured to use the measured change in vibration of the probe 104 to determine the at least one parameter of the eye.

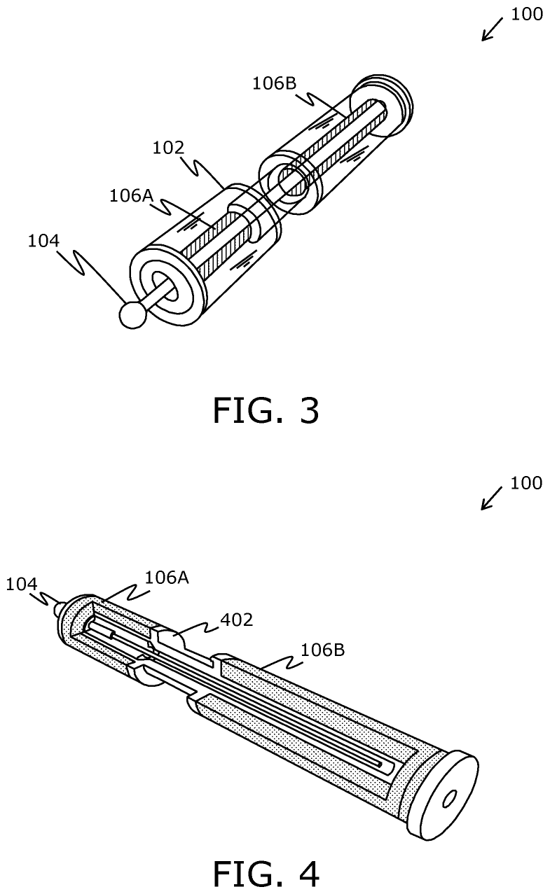

Referring to FIG. 2, there is shown a schematic illustration of the system 100 for measuring at least one parameter of an eye, in accordance with an exemplary embodiment of the present disclosure. As shown, the system comprises two coils 106A and 106B, wherein the two coils 106A an 106B surround a probe 104. Herein, the coils 106A and 106B are operable to release the probe 104 towards the surface of the eye and to retract the probe 104 into housing. Furthermore, the coils 106A and 106B are coupled to probe vibrating means 108, wherein the probe vibrating means 108 is a rnagnetostrictive oscillator. Specifically, the rnagnetostrictive oscillator 108 induces vibration in the probe 104. In this regard, the coil 106A forms a first coil L1 of the rnagnetostrictive oscillator 108 and the coil 106B forms a second coil L2 of the rnagnetostrictive oscillator 108.

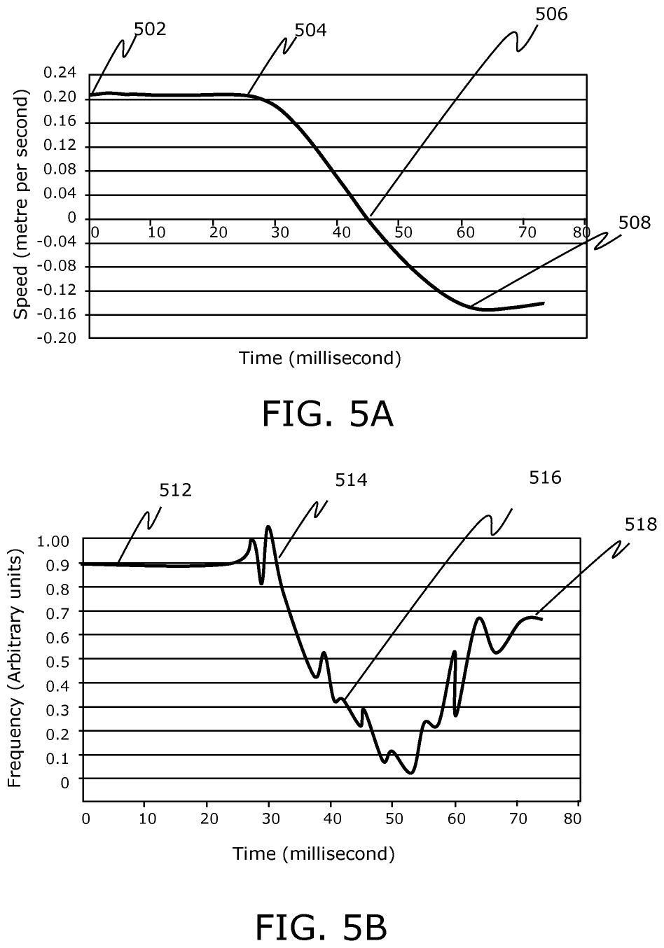

Referring to FIG. 3, there is shown a schematic illustration of the system 100 for measuring at least one parameter of an eye, in accordance with an exemplary embodiment of the present disclosure. As shown, the system 100 comprises a probe 104 detachably arranged within a housing 102. The probe 104 is surrounded by two coils 106A and 106B. Herein, the coils 106A and 106B are operable to release the probe 104 towards the surface of the eye and to retract the probe 104 into the housing 102.

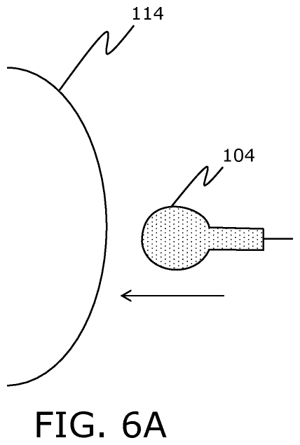

Referring to FIG. 4, there is shown a schematic illustration of the system 100 for measuring at least one parameter of an eye, in accordance with an exemplary embodiment of the present disclosure. As shown, the system 100 comprises a probe 104 detachably arranged within a housing 102. The probe 104 is surrounded by two coils 106A an 106B. Herein, the coils 106A and 106B are operable to release the probe 104 towards the surface of the eye and to retract the probe 104 into housing 102. Moreover, the coils 106A and 106B are arranged within a coil frame 402.

It may be understood by a person skilled in the art that the FIGS. 1, 2, 3 and 4 include simplified illustrations of the system 100 for measuring at least one parameter of an eye, for sake of clarity only, which should not unduly limit the scope of the claims herein. The person skilled in the art will recognize many variations, alternatives, and modifications of embodiments of the present disclosure.

Referring to FIG. 5A, there is shown a graphical representation 500 of change in speed of a probe with respect to change in time, in accordance with an embodiment of the present disclosure. The graphical representation 500 describes ideal movement of the probe based on speed. At 502, the probe is released from the housing. The probe moves towards the surface of the eye from the housing between 502 and 504 with predefined speed. Moreover, at 504, the probe impacts the surface of the eye. Furthermore, a decrease in speed of the probe is observed between 504 and 508. Herein, the probe exerts force on the surface of the eye between 504 and 506; and further at 506, speed of the probe reaches zero so as to stop further exertion of force on the surface of the eye. Moreover, the probe starts to retract after 506. At 508, the probe completely retracts from the eye to the housing. The retraction of the probe is due to compression of the at least one coil thereby further reducing the speed of the probe. The probe is then energized with sufficient speed for subsequent impact on the surface of the eye.

Referring to FIG. 5B, there is illustrated a graphical representation of the change of the frequency profile of the vibration upon the impact of the probe on the surface of the eye with respect to change in time, in accordance with an embodiment of the present disclosure, wherein the signals generated from motion, probe oscillation, and reflections are mixed. At 512, the probe is released from the housing. The probe moves towards the surface of the eye from the housing between 512 and 514 with a first initial value. Moreover, at 514, the probe impacts the surface of the eye. Furthermore, a decrease in frequency of the probe is observed after the probe has impacted the surface of the eye and starts moving back towards the housing 516 and when the probe has retracted to the housing, the probe achieves the second end frequency value 518.

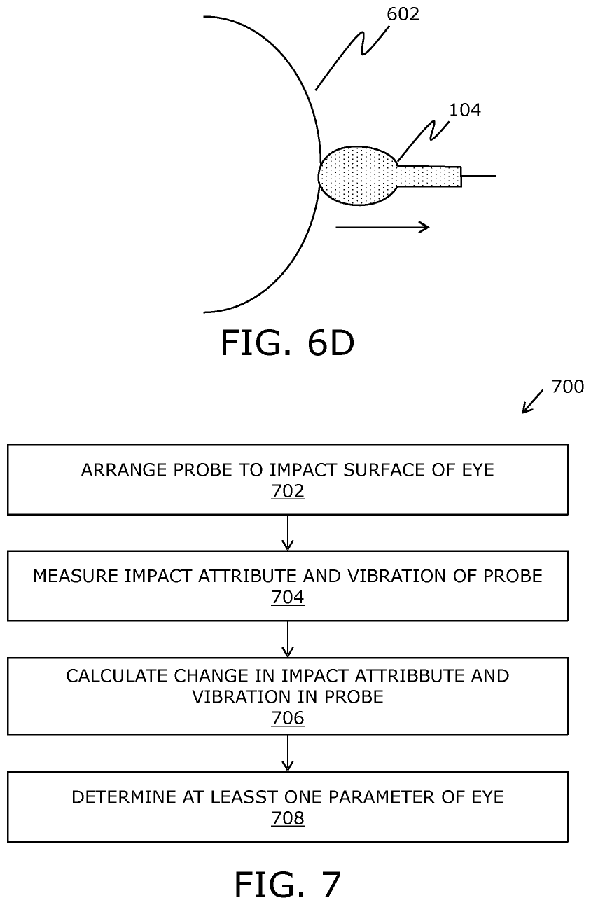

Referring to FIGS. 6A, 6B, 6C and 6D, there are shown schematic illustrations of movement of a probe 104 with respect to a surface 114 of an eye, in accordance with an embodiment of the present disclosure. Referring to FIG. 6A, there is shown a movement of the probe 104 from a housing (not shown) towards a surface 114 of the eye. Referring to FIG. 6B, there is shown an impact moment (namely, a moment of first contact) between the probe 104 and the surface 114 of the eye. Referring to FIG. 6C, there is shown a movement of the probe 104 inside the surface 114 of the eye. Herein, the probe 104 exerts force on the surface 114 of the eye. Referring to FIG. 6D, there is shown a retraction movement of the probe from the surface 114 of the eye towards the housing (not shown).

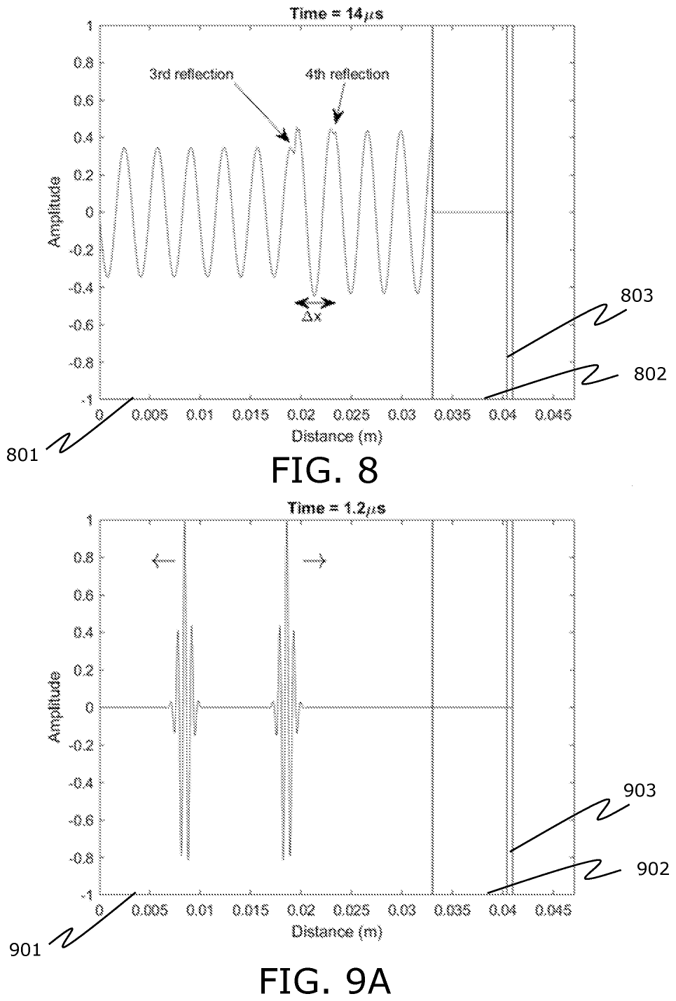

Referring to FIG. 7, there are shown steps of a method 700 for measuring at least one parameter of an eye, in accordance with an embodiment of the present disclosure. At a step 702, a probe is arranged to impact a surface of the eye with a predefined impact attribute and a predefined vibration. At a step 704, the impact attribute of the probe and vibration of the probe are measured during impact on the surface of the eye. At a step 706, a change in the vibration of the probe upon impact on the surface of the eye is calculated using the predefined impact attribute, the predefined vibration, the measured impact attribute, the measured vibration. At a step 708, the at least one parameter of the eye is determined using the change in the vibration of the probe.

The steps 702, 704, 706 and 708 are only illustrative and other alternatives can also be provided where one or more steps are added, one or more steps are removed, or one or more steps are provided in a different sequence without departing from the scope of the claims herein.

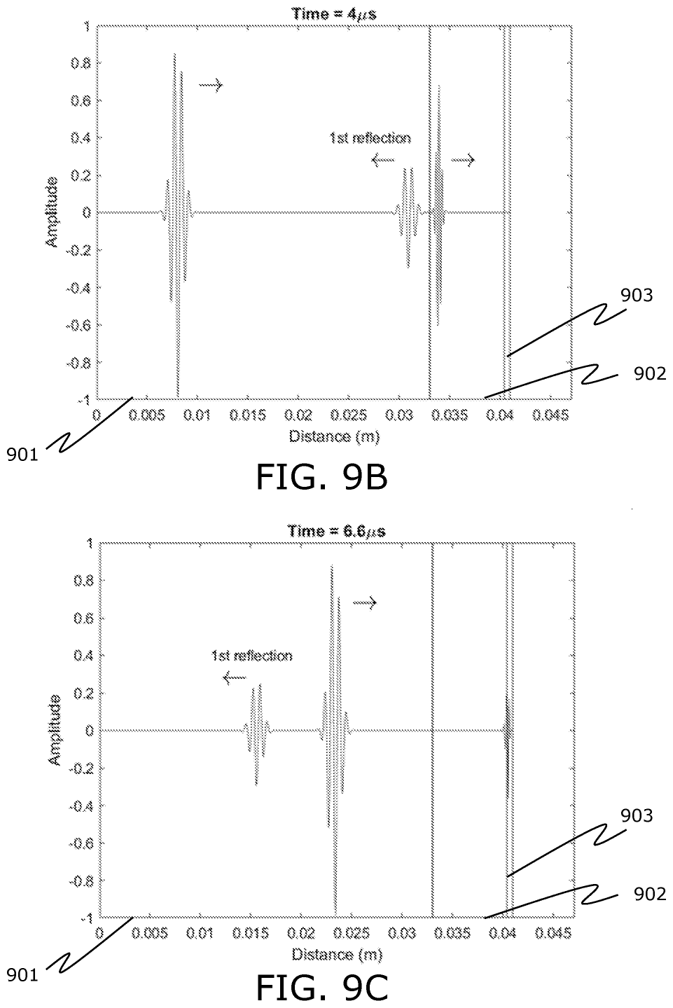

Referring to FIG. 8, there is illustrated a graphical representation of the waveform of standing wave vibration of the probe having a body of the probe 801 and a probe head 802 on the impact with the cornea 803, wherein the impact time is for example 14 μs, the length of the body of the probe 801 is for example 3.3 cm and the length of the probe head 802 is for example 0.7 cm.

Based on the propagation time difference between the 3rd and 4th reflections, the thickness of the cornea can be calculated by using the distance (ΔX) between the reflected 3rd and 4th waves and the speed of sound in the probe body 801. In this example the distance between the pulses in the probe body 801 is shown. The same can be turned into a time difference on the receiving coil when the pulse propagation speed on the probe is known. The distance (ΔX) can be measured by measuring the waveform of the vibration.

The excitation pulses are generated and the resulting reflections are measured from the probe body 801 by at least one coil. For example, the coils around the probe can generate a magnetostrictive ultrasonic pulse to the probe body 801. Reversibly, the vibration or pulse in the probe is detectable by the coils around it. The same applies to a pulsed excitation and a continuously oscillating resonator.

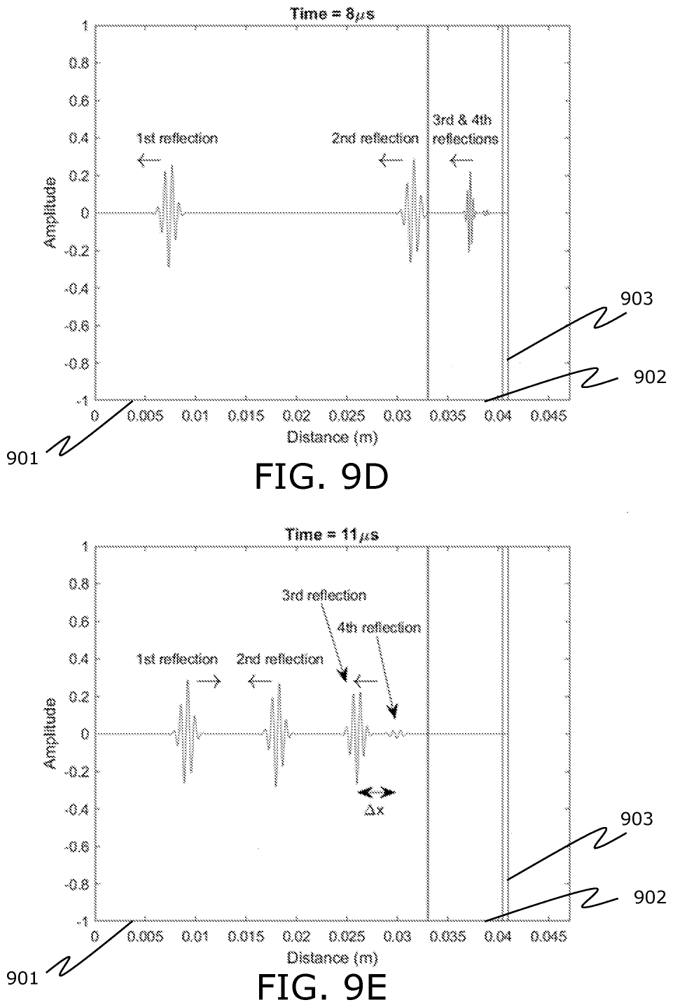

The figures FIG. 9A to FIG. 9E show the frequency wave propagation (of a pulsed vibration) in the probe during the impact of the surface of the eye, i.e. the surface of cornea, as shown in figure FIG. 8. The thickness of the cornea is determined by measuring the difference between of the reflected waves, more specifically by measuring the distance (physical or timing based) between the reflected third and fourth waves. In this embodiment, the body of the probe 901 is for example made of steel and the probe head 902 is for example at least partially made of biocompatible material (e.g. biocompatible material plastic).

On figure FIG. 9A during the 1.2 μs of the impact time a pulse induced in the probe leaves the middle part of the probe and proceeds propagating symmetrically through the body of the probe 901 in both directions, i.e. towards the housing and towards the probe head 902. On figure FIG. 9B during the 4 μs of the impact time a new induced pulse propagating through the body of the probe 901 towards the probe head 902. On the interface of the body of the probe 901 and probe head 902, part of the pulse is propagating by reflecting back, i.e. the first reflection of the pulse, from the interface of the body of the probe 901 and probe head 902 towards the housing and part of the pulse continues propagating in the probe head 902 towards the surface of the eye, i.e. surface of the cornea 903.

On figure FIG. 9C during the 6.6 μs of the impact time the 1st reflection continues propagating towards the housing, meanwhile the new pulse continues propagating towards the probe head 902 and the waves of the pulse reached to the probe head 902 continue propagating towards the surface of the eye, i.e. towards the surface of the cornea 903. On the interface of the body of the probe 901 and probe head 902, part of the new pulse is propagating by reflecting back, i.e. the second reflection of the pulse, from the interface towards the housing and part of the pulse continues propagating in the probe head 902 towards the surface of the eye. On the interface of probe head 902 and the surface of the eye 903 a first portion of the wave of the pulse in the probe head 902 is reflecting back, i.e. the third reflection and a second portion of the wave of the pulse in the probe head 902 is absorbing in the surface of the eye, i.e. in the surface of the cornea 903.

On figure FIG. 9D during the 8 μs of the impact time the 1st and the 2nd reflections of the waves of the pulses continue propagating through the body of the probe 901 towards the housing, the 3rd reflection and the reflection, i.e. fourth reflection, from the inner surface of the cornea 903, i.e. from the endothelium, continue propagating through the probe head 902 towards the interface of the of the body of the probe 901 and probe head 902.

On figure FIG. 9E during the 11 μs of the impact time the waves of 3rd reflection and 4th reflection of the pulses reflected from the inner (endothelium) and outer cornea (epithelium) propagate through the body of the probe 901 towards the housing. By measuring the distance ΔX between the 3^{rd} reflection and the 4^{th} reflection and considering the speed of sound in the cornea Ccornea=1640 m/s and the speed of sound in the probe for example Cprobe=5900 m/s, the thickness of the cornea CCT can be calculated:

CCT=½*Ccornea/Cprobe*ΔX

Using provided values in the example thickness is calculated to be CCT=½*1640/5900*0.004 m=555 um (micrometers) in the present example. The measurement of the distance can be done for example determining waveform by measuring an amplitude of vibration at any place (for example in the middle) in the probe as a function of time. The determined waveform can be used as a measurement of the distance ΔX.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural.

1. A system for measuring at least one parameter of an eye, the system comprising:
a probe detachably arranged within a housing, wherein the probe is operable to impact a surface of the eye with a predefined impact attribute;
at least one coil operable to maintain the probe within the housing, to release the probe towards the surface of the eye and to retract the probe into the housing;
a probe vibration means operable to induce vibration to the probe;
a measuring means for measuring a change in vibration of the probe upon impact on the surface of the eye; and
a controller configured to use the measured change in vibration of the probe to determine the at least one parameter of the eye.

2. The system of claim 1, wherein a frequency of the induced vibration of the probe is in a range of 0.5 kHz to 100 MHz.

3. The system according to claim 1, wherein the induced vibration of the probe is at least one of: a continuous vibration, a standing wave vibration, a pulsed vibration, a vibration comprising two or more vibration frequencies.

4. The system of claim 1, wherein the at least one parameter of the eye is thickness of cornea of the eye, pressure inside the eye, corneal water content.

5. The system of claim 1, wherein the probe vibration means comprises at least one of: a magnetostrictive oscillator, a piezoelectric oscillator, a transducer, an amplifier, a multivibrator.

6. The system of claim 1, wherein the impact attribute of the probe is at least one of: speed of the probe, kinetic energy of the probe.

7. The system of claim 1, wherein the measuring means comprises at least one of: a transducer, a speed sensor, a frequency sensor.

8. A method for measuring at least one parameter of an eye, the method comprising:
arranging a probe to impact a surface of the eye with a predefined impact attribute;
arranging the probe to impact the surface of the eye with a predefined vibration;
measuring impact attribute of the probe and vibration of the probe, during impact on the surface of the eye;
calculating, using at least one of: the predefined impact attribute, the predefined vibration, the measured impact attribute, the measured vibration, a change in the vibration of the probe, upon impact on the surface of the eye; and
determining, using the change in the vibration of the probe, the at least one parameter of the eye.

9. The method of claim 8, wherein a frequency of the induced vibration of the probe is in a range of 0.5 kHz to 100 MHz.

10. The method according to claim 8, wherein the induced vibration of the probe is at least one of: a continuous vibration, a standing wave vibration, a pulsed vibration, a vibration comprising two or more vibration frequencies.

11. The method of claim 8, wherein the at least one parameter of the eye is any one of: thickness of cornea of the eye, pressure of the eye, corneal water content.

12. The method of claim 8, wherein the impact attribute of the probe is at least one of: speed of the probe, kinetic energy of the probe.
